(19) United States
(12) Patent Application Publication
(10) Pub. No.: US 2006/0110740 A1
(43) Pub. Date: May 25, 2006

(54) Use of sendai virus as a human parainfluenza vaccine

(76) Inventors: Julia Hurwitz, Germantown, TN (US); Christopher Coleclough, Germantown, TN (US); Karen Slobod, Memphis, TN (US); Allen Portner, Bartlett, TN (US)

Correspondence Address: ST. JUDE CHILDREN'S RESEARCH HOSPITAL;OFFICE OF TECHNOLOGY LICENSING, 332 N. LAUDERDALE, MEMPHIS, TN, 38105, US

(21) Appl. No.: 10/540,367

(22) PCT Filed: Jan. 12, 2004
(86) PCT No.: PCT/US04/00635

§ 371 Date: Jun. 23, 2005

Related U.S. Application Data
(60) Provisional application No. 60/441,369, filed on Jan. 20, 2003.

(51) Int. Cl. C12Q 1/70; C12Q 1/68

(52) U.S. Cl. 435/6; 435/5

(57) ABSTRACT

Immunogenic compositions to protect against human parainfluenza (hPIV) virus infection are provided. The compositions comprise an unmodified Sendai virus that can be safely administered to human subjects, particularly young children, to protect against symptoms and health risks associated with hPIV infection.

### CROSS REFERENCE TO RELATED APPLICATIONS

This application claims priority to U.S. Provisional Application No. 60/441,369 filed Jan. 20, 2003.

### GOVERNMENT INTERESTS

This invention was made in part with U.S. Government support under National Institutes of Health grant nos. AI11949, R01-CA57419, P30-CA21765, P01-AI31596 and RR00164-34 and was also supported by funds from the American Lebanese Syrian Associated Charities (ALSAC). The U.S. Government may have certain rights in this invention.

### FIELD OF THE INVENTION

This invention relates to methods to protect humans from parainfluenza virus infection.

### BACKGROUND

Human parainfluenza virus type-1 (PIV-1) is a major cause of hospitalization of infants due to “croup”. As a result, a number of attempts have been made to generate an effective vaccine against this virus. These include attempts to use bovine PIV-3 (van Wyke Coelingh et al., J. Infect. Dis. 157: 655 (1988)), attenuated forms of parainfluenza virus (U.S. Pat. No. 6,410,023), purified PIV or respiratory syncytial virus proteins (U.S. Pat. Nos. 6,180,398; 6,165,774), chimeric PIV proteins (U.S. Pat. No. 6,225,091;), or combinations of the above (U.S. Pat. No. 5,976,552). However, these approaches have not yet yielded a safe and effective parainfluenza virus vaccine.

The possibility of using murine PIV-1 (Sendai virus) to protect against human PIV-1 (hPIV-1) has also been suggested. See Gorman et al., “The hemagglutinin-neuramimidase glycoproteins of human parainfluenza virus type 1 and Sendai virus have high structure-funtion similiarity with limited antignenic corss-reactivity”, Virology 175: 211-221 (1990); Sangster, M. et al., “Human parainfluenza virus type 1 immunization of infant mice protects from subsequent sendai virus infection”, Virology 212: 13-19 (1995); Hurwitz, J. L. et al., “Intranasal Sendai virus vaccine protects African green monkeys from infection with human parainfluenza virus-type one”, Vaccine 15(5): 533-540 (1997). However, experts in the field have rejected this method due to concerns that

(i) Sendai virus may cause disease in humans, and (ii) Sendai virus may not elicit cross-reactive antibodies toward human PIV-1. Skiadopolous, MH. et al., “Sendai virus, a murine parainfluenza virus type 1, replicates to a level similar to human PIV1 in the upper and lower respiratory tract of African green monkeys and chimpanzees”, Virology 297: 153-160 (2002).

### SUMMARY OF THE INVENTION

The present invention provides for the use of Sendai virus to protect against parainfluenza (PIV) infection, particularly human parainfluenza-1 (hPIV-1) infection. According to the present invention Sendai virus, preferably murine Sendai virus, may be safely administered to a subject to generate an immune response that will protect the subject from PIV infection.

Preparation of unmodified Sendai virus in a form suitable for administration as an immunogenic composition or vaccine is taught by the present invention.

In related aspects, the invention provides a method for stimulating the immune system to elicit an immune response against PIV in a mammalian subject. The method comprises administering a formulation of an immunologically sufficient amount of Sendai virus in a physiologically acceptable carrier and/or adjuvant. In one embodiment, the immunogenic composition is a vaccine comprised of a purified Sendai virus. The vaccine can be formulated in a dose of 1×10⁵-1×10⁸ PFU (plaque forming units) of Sendai virus. The vaccine elicits an immune response against any hPIV-1 species. Preferably the immunogenic composition is administered to the upper respiratory tract, e.g., by spray, droplet or aerosol.

### DETAILED DESCRIPTION OF THE INVENTION

Definitions:

Sendai virus: Sendai virus is a mouse parainfluenza virus which is the murine homologue of hPIV-1. All Sendai virus (murine parainfluenza virus type 1) strains or variants are related by (a) antigenic relatedness and amino acid and nucleotide sequence identity, (b) conserved sequence motifs at the 3′ and 5′ ends of the genome and at the ends of each gene, (c) conserved trinucleotides in intergenic regions, (d) identical RNA editing details. Sendai virus regardless of the source is expected to be effective against any member of the hPIV-1 species. The complete genomic sequence of a typical Sendai virus strain is available at genbank accession no. NC_{—}001552.

Human Parainfluenza (hPIV): The human paramyxoviruses exist as four known species, known as types 1-4. All human parainfluenza type 1 viruses contain common features which define this species: (a) antigenic relatedness and amino acid and nucleotide sequence identity, (b) conserved sequence motifs at the 3′ and 5′ ends of the genome and at the ends of each gene, (c) conserved trinucleotides in intergenic regions, (d) identical RNA editing details. Sendai virus is expected to be effective against any member of the hPIV-1 species.

Immunogenic composition: A composition designed to elicit an immune response (a B-cell and/or T-cell response) when administered to a subject. The immunogenic composition will contain the selected immunogen(s) and may also include allantoic fluid and a pharmaceutically acceptable carrier or adjuvant (e.g. alum, aluminum hydroxide), or an immunostimulant (e.g. IL-2). By “immunogen” is meant any virus-related gene, protein or peptide intended to elicit a B and or T cell response. Exemplary pharmaceutically acceptable carriers include, but are not limited to, sterile pyrogen-free water, sterile pyrogen-free physiological saline solution and phosphate buffered saline. Sendai virus proteins or recombinant reagents expressing Sendai virus proteins may also be used.

An “immunological response” is the development in the host of a cellular and/or antibody-mediated immune response to the immunogenic composition or vaccine of interest. Usually, an immunological response includes but is not limited to one or more of the following effects: the production of antibodies, the activation of B cells, helper T cells, suppressor T cells, and/or cytotoxic T cells (alpha-beta or gamma-delta) to an antigen or antigens included in the immunogenic composition or vaccine of interest. Preferably, the host will display either a therapeutic or protective immunological response such that resistance of the subject to infection by PIV will be enhanced and/or the clinical severity of symptoms associated with PIV infection will be reduced. Such protection will be demonstrated by either a reduction or lack of symptoms normally displayed by an infected host and/or a quicker recovery time and/or a shortened period of virus shedding.

Vaccine: An immunogenic composition that is administered to protect a host from infection by a target virus or pathogen.

Description:

The present invention provides for the use of Sendai virus to protect against parainfluenza (P) infection, particularly human parainfluenza (hPIV) infection. Administration of immunogenic compositions containing Sendai virus elicits production of an immune response that is protective against upper or lower respiratory tract disease, such as pneumonia and bronchiolitis when the subject is subsequently infected with PIV.

Sendai virus useful in the present invention may be obtained from a variety of host sources, including mice trapped from the wild as well as infected laboratory mice. Sendai virus can be isolated and purified from a host animal using conventional techniques. Isolated Sendai virus strains are also available from various academic laboratories including St. Jude Children's Research Hospital (laboratory of Dr. A. Portner), the NIH (laboratory of Dr. B. Murphy); Northwestern University, Chicago (laboratory of Dr. R. Lamb); Toyama Institute of Health, Toyama, Japan (laboratory of Dr. Y. Nagai); and the University of Geneva, Switzerland (laboratory of Dr. D. Kolakofsky). The technology of reverse genetics provides additional means to generate Sendai virus strains, variants and mosaic infectious Sendai virus consisting of gene elements from different strains and variants (Garcin D. et al., EMBO J 14: 6087-6094 (1995), Kato A. et al, Genes and Cells 1: 569-579(1996)).

The host to which the Sendai virus may be administered can be any human which is susceptible to infection by PIV or a closely related virus and which host is capable of generating a protective immune response to the antigens of the vaccine strain. Accordingly, the invention provides methods for creating vaccines for a variety of human uses. Individuals who are considered to be particularly susceptible to hPIV infection, such as neonates, infants and small children, are preferred subjects for the vaccine as taught herein. Thus preferred subjects for administration of the compositions of the invention include neonates, infants and small children ranging in age from less than 1 year old to about 10 years old, more preferably from 1 month to 5 years old, and more preferably from 6 months to 1 year old.

Sendai virus may be prepared for use in an immunogenic composition of the invention using standard techniques. For example, Sendai virus may be grown in chicken eggs by infection of allantoic fluid or tissue culture cells. Sendai virus is grown in the allantoic cavity of 10-day-old embryonated eggs. Virus is concentrated and purified by differential centrifugation and sedimentation through sucrose gradients. See Portner, A. et al., J. Virol. 13: 298-304 (1974); Thompson S. D. et al., J. Virol. 62: 4653-4660, (1988). Sendai virus can also be grown in a wide variety of primary and continuous cell monolayer cultures derived from avian and mammalian sources including human and non human primates. For example, primary chick embryo lung cells (Portner, A and Kingsbury, D. W., Virology 47: 711-725 (1972)); monkey kidney cells (Portner, A et al., J. Virol. 13: 298-304 (1974)) and HeLa cells.

Sendai virus may also be propagated in a variety of cultured mammalian cells. Sendai virus may be produced using primary trypsinized cells, including cells from monkey kidneys, and the kidneys of rabbits and hamsters. Sendai virus may also be produced in continuous cell lines such as MDCK cells (Frank et al., J. Clin. Microb. 10:32-36 (1979); Schepetink & Kok, J. Virol. Methods 42:241-250 (1993)), African green monkey kidney (Vero) cells and baby hamster kidney (BK-21). The latter two cell lines have been approved and certified by the World Health Organization (WHO) for production of human vaccines. Growth in continuous cell lines may be preferred because viruses tend to retain their antigenic characteristics when grown this way. Katz et al., Virology 165: 446-456 (1988); Robertson et al., Virology 179:35-40 (1990); Katz et al., J. Infect. Dis. 160:191-198 (1989); Wood et al., Virology 171:214-221 (1989).

According to the teachings of the present invention, the propagated Sendai virus does not have to be inactivated prior to use as a vaccine. It simply must be placed in a form suitable for administration to a subject through conventional techniques.

Immunogenic compositions comprising Sendai virus as described herein may be administered via aerosol, droplet, oral, topical or other route. Administration of live Sendai virus may be carried out by any suitable means, including both parenteral injection (such as intraperitoneal, subcutaneous, or intramuscular injection) and more preferably by topical application of the virus (typically carried in the pharmaceutical formulation) to an airway surface. Topical application of the virus to an airway surface can be carried out preferably by intranasal administration (e.g. by use of dropper, swab, or inhaler which deposits a pharmaceutical formulation intranasally). Topical application of the virus to an airway surface can also be carried out by inhalation administration, such as by creating respirable particles of a pharmaceutical formulation (including both solid particles and liquid particles) containing the virus as an aerosol suspension, and then causing the subject to inhale the respirable particles. Methods and apparatus for administering respirable particles of pharmaceutical formulations are well known, and any conventional technique can be employed.

Upon administration, the immune system of the host responds by producing antibodies specific for PIV virus proteins, e.g., F and HN glycoproteins. As a result of administration with an immunogenically effective amount of Sendai virus produced as described herein, the host becomes at least partially or completely immune to PIV infection, or resistant to developing moderate or severe PIV infection, particularly of the lower respiratory tract.

The immunogenic and vaccine compositions of the invention are administered to a host susceptible to or otherwise at risk for PIV infection to enhance the host's own immune response capabilities. Such an amount is defined to be an “immunogenically effective dose.” In this use, the precise amount of the composition to be administered within an effective dose, and the timing and repetition of administration, will be determined based on the patient's state of health, age and weight, the mode of administration, the nature of the formulation, etc. Dosages will generally range from about 1×10⁵-1×10⁸ plaque forming units (PFU) or more of virus per host, more commonly from about 5×10⁵-5×10⁷ PFU virus per host. In any event, the Sendai virus compositions should provide a quantity of Sendai virus of the invention sufficient to effectively stimulate or induce an anti-PIV immune response, e.g., as can be determined by complement fixation, plaque neutralization, enzyme-linked immunosorbent assay, and/or other measures of antibody binding, among other methods. Preferably the immunogenic and vaccine compositions should provide a quantity of Sendai virus of the invention sufficient to effectively protect the host patient against serious or life-threatening PIV infection.

In some instances it may be desirable to combine the PIV vaccines of the invention with vaccines which induce protective responses to other agents, particularly other childhood viruses.

In neonates and infants, multiple administration may be required to elicit sufficient levels of immunity. Administration should begin within the first year of life, and at intervals throughout childhood, such as at two months, six months, one year and two years, as necessary to maintain sufficient levels of protection against native (wild-type) PIV infection. Similarly, adults who are particularly susceptible to repeated or serious PIV infection, such as, for example, health care workers, day care workers, family members of young children, the elderly, individuals with compromised cardiopulmonary function, may require multiple immunizations to establish and/or maintain protective immune responses. Levels of induced immunity can be monitored by measuring amounts of neutralizing mucosal/secretory and serum antibodies, and dosages adjusted or vaccinations repeated as necessary to maintain desired levels of protection.

Levels of induced immunity provided by the vaccines and immunogenic compositions of the invention can be monitored by measuring amounts of neutralizing mucosal/secretory and serum antibodies. Based on these measurements, dosages can be adjusted or administration repeated as necessary to maintain desired levels of protection.

### EXAMPLES

### Example 1

### Intranasal Sendai Virus Vaccine Protects African Green Monkeys from Infection with Human Parainfluenza Virus-Type One (hPIV-1)

Summary

Human parainfluenza virus-type 1 (hPIV-1) infections are a common cause of “croup” and hospitalizations among young children. Here we address the possibility of using the xenotropic Sendai virus as a vaccine for hPIV-1. Sendai virus was administered to six African green monkeys (Cercopithecus aethiops) by the intranasal route, A long lasting virus-specific antibody response was elicited, both in the serum and nasal cavity. Sendai virus caused no apparent clinical symptoms in the primates, but live virus was shown to persist in the nasal cavity for several days after inoculation. No virus persisted when a second dose of Sendai virus was administered on day 126 after the initial priming. Animals were challenged with hPIV-1 intranasally on day 154. All six vaccinated animals were fully protected from infection while six of six control animals were infected with hPIV-1. The antibody and protective responses induced by Sendai virus immunizations proved to be greater than those induced by hPIV-1. These results demonstrate that unmanipulated Sendai virus is an effective vaccine against hPIV-1 in a primate model and may constitute a practical vaccine for human use.

Introduction

Here we describe the testing of Sendai virus as an hPIV-1 vaccine in African green monkeys. Results demonstrate that the intranasal administration of Sendai virus to these primates provides absolute protection against hPIV-1.

Materials and Methods

Animals/Handling

African green monkeys were feral caught, and housed at the Tulane primate center. Animals were anesthetized with ketamine (10 mg/ml) prior to handling. Throat swabs were taken from test animals on day 0, immediately prior to animal inoculation with hPIV-1 or Sendai virus. Nasal swabs were taken after day 0 for the measure of virus and antibody titers. Swabs were placed in 0.5 ml minimum essential medium (MEM) plus 5% fetal calf serum (FCS) and stored at −70 degrees C. Vials were vortexed prior to sampling for virus and antibody assays.

Virus Preparation and Immunizations

Sendai virus (Enders strain) was egg grown and was titered at 7.6×10⁸ EID₅₀/ml. Human PIV-1 (strain C35 from the American type culture collection, ATCC) was prepared by infecting confluent monolayers of LLC-MK₂ cells with virus. Supernatants were stored at −70° C. at a titer of 10⁹ plaque forming units (pfu)/ml. All immunizations were performed by the intranasal route with virus in 0.5-1 ml final volume.

Measurement of Sendai Virus and hPIV-1 in Swab Samples

Sendai virus assay: Swab samples were vortexed and serial dilutions were made in PBS containing 0.1% bovine serum albumin (BSA), 100 U/ml penicillin, and 100 μg/ml streptomycin. Samples were inoculated into the allantoic cavity of 10-day-old embryonated eggs (3 eggs/dilution). After incubation at 35° C. for 48 h, allantoic fluid from each egg was tested for the presence of virus by the hemagglutination (HA) assay using chicken red blood cells (RBC) in a final volume of 50 μl.

hPIV-1 assay: plague count: LLC-MK₂ cells were grown to confluency in 60 mm plates in complete medium (MEM (Gibco, Grand Island, N.Y.), 0.2% NaHCO₃, 2 mM glutamine, and 50 μg/ml gentamicin (BioWhittaker, Walkersville, Md.)) with 5% fetal calf serum (FCS). Plates were washed twice in PBS/calcium/magnesium. Nasal swab samples were diluted in 0.15% bovine serum albumin (BSA) in PBS/calcium/magnesium and gentamicin and plated onto washed LLC-MK2 cells (100 μl/well). After a 45 minute absorption at room temperature, cells were overlayed with 6 ml complete medium, plus 0.15% BSA, supplemental vitamins and amino acids, 5 μg/ml acetylated trypsin, and 0.9% agarose (electrophoresis grade, BRL, Gaithersberg, Md.). After the agarose was set, plates were inverted and incubated at 34° C. in a 5% CO₂ incubator. After 5-7 days, plates received a second overlay (5 ml), similar to the first, but with 5% FCS instead of BSA, 0.0035% neutral red, and no trypsin supplement. Plates were incubated for an additional 2-3 days and plaques were counted.

hPIV-1 assay: virus amplification followed by HA analysis: Confluent cultures of LLC-MK₂ cells in 24-well flat bottomed plates were washed and inoculated with 0.1 ml test swab samples diluted 1:10 in 0.15% BSA in PBS/calcium/magnesium with antibiotic. Plates were incubated for 1 hour at room temperature with rocking at regular intervals. The samples were removed and wells were fed with 0.15% BSA in complete medium with 0.5 μg/ml trypsin. Incubation continued for six days at 34° C., 5% CO₂. At the completion of the six day period, culture supernatants were sampled. Serial dilutions of sample supernatants were tested for HA activity with chicken RBC in a 50 μl test volume, incubated at 4° C. for 30 minutes.

Enzyme-linked immunosorbent assay (ELISA): Sucrose-banded hPIV-1 or Sendai (Enders strain) viruses were dissociated with disruption buffer (0.05M Tris-HCl, 0.6M KCl and 0.5% Triton-X 100 (pH7.8)). Disrupted virus was diluted in PBS, pH 7.2, to 10 μg/ml, and 50 μl volumes added to the wells of 96-well Nunc-ImmunoMaxiSorp plates. ELISAs were completed as described previously (Smith, F. S. et al., “Age-related development of human memory T-helper and B-cell responses towards parainfluenza virus-type 1”, Virology 205: 453-461 (1994)) by blocking plates and then incubating with test and control samples. Assays were developed either with alkaline-phosphatase-conjugated anti-human IgG (H+L) antibody, or horse radish peroxidase-conjugated goat anti-human IgA (α) antibody from BioRad (Hercules, Calif.). Subsequent incubation with p-nitrophenyl phosphate (Sigma, St. Louis, Mo.) for the alkaline phosphatase conjugated antibody, or ABTS (Boehringer Mannheim, Indianapolis, Ind.) for the horse radish peroxidase conjugated antibody, initiated the color reaction. The absorbance of each well was read at 405 nm using a microplate reader (Model 3550, Bio-Rad).

Hemagglutination inhibition (HAI) assay: Serum (20 μl) was added to 80 μl receptor destroying enzyme (RDE) of Vibrio cholerae (Center for Disease Control, Biological Reagents Section, Atlanta Ga.; reconstituted and diluted in calcium saline as recommended by distributors). After overnight incubation at 37° C., 60 μl of sodium citrate was added before heating at 56° C. for 30 min. The final volume was brought to 200 μl with phosphate buffered saline (PBS). Hemagglutination inhibition titer determinations were made using 25 μl volumes of serially diluted, RDE-treated serum samples, 25 μl of four agglutinating doses of either Sendai virus or hPIV-1, and 50 μl of 0.5% chicken red blood cells.

Neutralization assay: Plaque assays were performed as described above. Virus was used at a titer yielding countable plaques (approximating 40-100 plaques/plate). Prior to plating, virus was incubated with an equal volume of a test or control serum sample for one hour at room temperature. Plaques were processed and counted as described above. Serum dilutions were considered positive for neutralization when plaque numbers were reduced by ≧85%.

Results

The present study was initiated to assess the safety and efficacy of a PIV-type 1 from mice (Sendai virus) as a vaccine in primates. As outlined in Table 1, the study involved 18 African green monkeys. The first group of six animals was inoculated with Sendai virus (7.6×10⁷ EID₅₀) intranasally on two occasions separated by 126 days, and this group subsequently received hPIV-1 (10⁶ pfu) on day 154. Human PIV-1 was administered to a second group of six monkeys, with pairs of animals receiving 10⁹, 10⁷ or 10⁵ pfu apiece, followed 154 days laterby a second inoculation with hPIV-1 at a dose of 10⁶ pfu per animal. One animal (N847) did not receive the second dose of hPIV-1 because it had developed a leg disorder unrelated to the vaccine trial. A control group of six monkeys received 1 ml allantoic fluid intranasally as a 1:5 dilution, and was subsequently challenged with 10⁶ pfu of hPIV-1. Animals were monitored for clinical symptoms, and serum samples and nasal swabs were obtained following each viral challenge. We describe the results of Sendai virus priming first, then of hPIV-1 priming.

Sendai Virus Persists in the Nasal Cavity for Several Days Following Primary but not Secondary, Inoculation.

Of the six animals inoculated with Sendai virus intranasally (group 1), all were sampled by nasal swab on days 1, 2, 3, 4, 5, 7, 8 and 9 following the first inoculation for evidence of live virus. After the second inoculation with Sendai virus (day 126), nasal swabs were taken from the same animals on days 1, 2, 3, 4, 5 and 7. For each swab sample, three eggs were first inoculated with 0.1 ml of a 1:5 dilution of nasal swab sample. Swabs taken after the first inoculation yielded virus on days 1-5 with virus clearance evident in all cases by days 7-9. After the second inoculation, no virus could be identified in any animal on day 2 or thereafter.

To determine whether the virus simply persisted longer after the first versus second inoculations, or whether there was active growth of the virus, serial dilutions of nasal swabs taken after the first inoculation were prepared for virus testing in eggs. A comparison of virus titers in day 1 nasal swabs with swabs taken on subsequent days demonstrated a total amplification of approximately 100× (occurring by days 24) followed by a steady reduction of virus prior to full clearance. These results reflected the active growth of the virus. Animals were examined for clinical symptoms including rhinorrhea, diarrhea, coughing, sneezing, rapid respiration, lethargy, restricted movement, loss of appetite and dizziness. No clinical symptoms were evident in any animal following the first or second inoculation with Sendai virus.

TABLE 1
Protocol for inoculation of test animals

| | | Treatment | Treatment | Treatment |
|---|---|---|---|---|
| Group | Animals | day 0 | day 126 | day 154 |
| 1 | M627 | Sendai | Sendai | Challenge |
| | | virus | virus | hPIV-1 |
| | M628 | 7.6 × 10⁷ | 7.6 × 10⁷ | 10⁶ p.f.u. |
| | | EID₅₀ | EID₅₀ | |
| | N836 | | | |
| | N837 | | | |
| | N842 | | | |
| | N845 | | | |
| 2 | N839 | hPIV1-10⁹ p.f.u. | None | Challenge |
| | N621 | | | hPIV-1 |
| | N843 | hPIV1-10⁷ p.f.u. | | 10⁶ p.f.u. |
| | N844 | | | |
| | N841 | hPIV-1-10⁵ p.f.u. | | |
| | N847 | | | |
| 3 | K089 | None | Allantoic fluid | Challenge |
| | | | | hPIV-1 |
| | M396 | | | 10⁶ p.f.u. |
| | P778 | | | |
| | P783 | | | |
| | P790 | | | |
| | P800 | | | |

Sendai Virus Induces a Strong, Durable PIV-Specific Antibody Response.

The six animals in group 1 (see Table 1) were primed by an intranasal inoculation of Sendai virus, boosted with the same virus 126 days later, and challenged with hPIV-1 after an additional one month (28 day) period. Serum samples were taken throughout the period of immunization and challenge. FIG. 1 shows the results of an ELISA with serum samples from all six Sendai virus-primed animals (group 1, solid symbols and X) and six control animals (group 3, clear symbols and +). The Sendai virus-primed animals showed an enhancement of virus-specific serum antibody until days 10-14, after which peak levels of antibody were retained throughout the course of the experiment As expected, virus-specific antibody was exhibited in the control animals (group 3) only after the hPIV-1 inoculation on day 154 (see Table 1).

To determine whether antibody demonstrated functional capacity in vitro serum samples from the Sendai virus-primed (group 1) animals were tested two weeks prior to the challenge with hPIV-1 for HAI and neutralization activity. For the HAI assay, serum was first diluted 1:10 and then serial 1:2 dilutions were prepared for testing. The end-point dilutions yielding HAI with Sendai virus ranged from 1:320-1:1280, whereas the end-point dilutions yielding HAI with hPIV-1 ranged from 1:40-1:80. Neutralization assays on hPIV-1 were performed with a 1:50 serum dilution. Positive neutralization function, scored as a ≧85% inhibition of hPIV-1 plaques, was evident in sera from five of the six animals in group 1. Sera from control animals (group 3) showed no HAI or neutralization activity.

Sendai Virus Induces PIV-Specific Antibody Responses in the Nasal Cavity.

Nasal swabs were taken from animals after each immunization and the hPIV-1 challenge. Whole immunoglobulin and Sendai virus-specific immunoglobulin were measured. Whole immunoglobulin was readily detectable in all nasal swabs at roughly comparable levels. Swabs from the Sendai virus-primed animals were positive for Sendai-virus specific antibody as early as day 7 after the first immunization. This antibody was sustained throughout the experimental course.

Sendai Virus Inoculations Protect African Green Monkeys from Subsequent Infection with hPIV-1

All six Sendai virus primed and boosted animals (group 1) were challenged with a dose of 10⁶ pfu hPIV-1 intranasally, as were six control animals (group 3). Nasal swabs were taken for 8 days thereafter and for the sampling of hPIV-1 by the inoculation of LLC-MK₂ monolayers.

All samples from Sendai virus primed (group 1) and control animals (group 3) were cultured for six days on LLC-MK2 cells and assayed for HA. Virus could not be recovered from day 1 swabs from any animal. By day 2, however, virus was identified in all six control animals, demonstrating the active growth of hPIV-1. In contrast, virus was not detectable in any of the Sendai virus-vaccinated animals. Thus full protection against hPIV-1 was achieved in Sendai virus immunized animals without any apparent clinical symptoms.

hPIV-1 Induces an Antibody Response, but Does not Provide Absolute Protection in African Green Monkeys.

Six naive African green monkeys (group 2) were placed in three groups of two and given intranasal doses of hPIV-1 of titers 10⁹ (high), 10⁷ (medium) and 10⁵ (low) pfu respectively. Animals were challenged with hPIV-1 154 days later (see Table 1). Antibodies were induced in all six animals. Titers did not achieve levels comparable to those of Sendai virus-primed animals. Serum antibody titers dropped in hPIV-1 inoculated animals and reached levels close to background in some animals by 133 days after inoculation. Interestingly, the absolute antibody titer did not reflect the dose of virus used in the inoculation. In fact, the animal with the lowest serum antibody titer (M621) was from the group of two animals given the highest dose of hPIV-1. Apparently, all doses were infectious and therefore comparable in their ability to elicit immunity.

To determine whether the apparently weak response in hPIV-1-primed animals was due to the fact that ELISAs were run on Sendai virus-coated plates, serum samples from animals that had been initially inoculated with either Sendai virus or hPIV-1 were tested in parallel in ELISAs with Sendai virus and hPIV-1 coated plates. In every tested case, antibodies cross-reacted between the two viruses; the titration curves for antibodies were similar regardless of whether the plate was coated with Sendai virus or hPIV-1. This evidence of reciprocal cross-reactivity was reminiscent of previous studies with mouse and human samples (Ryan K W, Murti K G, Portner A. Localization of P protein binding sites on the Sendai virus nucleocapsid. J Gen Virol 71:997-1000, 1990; Henrickson J K, et al., “Neutralization epitopes of human parainfluenza type 3 are conformational and cannot be imitated by synthetic peptides”, Vaccine 9:243-249, 1991; Ryan K W, et al., “Two noncontiguous regions of Sendai virus P protein combine to form a single nuleocacpsid binding domain”, Virology 180:126-134, 1991). Thus, the relatively weak response in hPIV-1 primed animals as compared to Sendai virus primed animals was evident with ELISAs performed either on Sendai virus or hPIV-1 coated plates.

To examine the functional activity of serum antibody after hPIV-1 infection, serum samples from infected animals (group 2) were tested two weeks prior to the second exposure to hPIV-1, for HAI and neutralization activity. For the HAI assay, serum was first diluted 1:10 and then serial 1:2 dilutions were prepared for testing. The end-point dilutions yielding HAI on hPIV-1 ranged from 1:80-1:320, whereas no HAI with Sendai virus could be demonstrated. Neutralization assays were performed with 1:50 serum dilutions. Positive neutralization function, scored as a ≧85% inhibition of hPIV-1 plaques, was evident in sera from five of five tested animals (serum was not taken from animal N847, due to an unrelated leg ailment). Sera from control animals showed no HAI or neutralization activity.

Measures of virus-specific antibody in the nasal swabs of hPIV-1-primed animals showed that, as was the case for serum antibody, the antibody was relatively weak in magnitude as compared to that from Sendai virus-primed animals.

Nasal swabs were sampled for virus on eight consecutive days after the first and second hPIV-1 inoculation of group 2 animals. Virus was amplified for six days in tissue culture and assayed for HA activity. All animals were clearly infected following the first inoculation with hPIV-1. However only one animal was infected after the second dose of hPIV-1. The one animal (M621) with evidence of hPIV-1 growth after the second inoculation was that with the lowest serum antibody response. Again, this animal was from the group of two animals that had received the highest dose of hPIV-1 during the “priming” stage.

Nasal swabs were also assayed for virus prior to amplification by tissue culture. In this case, samples taken from animal N844 after the first hPIV-1 exposure were tested on LLC-MK₂ cells with a plaque assay. The swab material (originally washed from the cotton into 0.5 ml collecting medium) was diluted 1:10 in antibiotic-containing medium and then plated on LLC-MK2 monoloayers with 100 μl sample per well. Plaque counts per well for samples taken on days 1, 2, 3, 4 and 5 after the first hPIV-1 infection averaged respectively 2, 186, 41, 40 and 9. Thus, the highest titer was from the day 2 swab taken after first infection, approximating 9,300 plaques per 0.5 ml collecting medium.

Sendai Virus Priming Induces Intranasal Antibody of the IgA Isotype

As one explanation for the better protection elicited by Sendai virus than by hPIV-1, one might suspect that the nasal IgA isotype may be superior in the Sendai virus-primed animals. The nasal swabs that were taken from all animals immediately prior to challenge (day 154) were therefore tested for IgA isotype. The results showed that it was indeed the case that PIV-specific IgA isotype appeared in all Sendai-virus primed animals, but did not exceed background levels in animals primed with hPIV-1.

Discussion

The present example describes the absolute protection provided to African green monkeys against hPIV-1 by the intranasal administration of Sendai virus vaccine. The Sendai virus vaccine caused no apparent clinical symptoms, but grew in the nasal cavity for several days. A strong, durable PIV-specific serum antibody response was generated that persisted throughout the course of the experiments. Intranasal IgA antibody isotype was also evident. Six of six animals given a Sendai virus prime (day 0) and boost (day 126) were protected from a subsequent challenge with hPIV-1 (day 154), while six of six control animals were infected.

In parallel with the study of Sendai virus, one group of animals was given one dose of hPIV-1 (without Sendai virus) and challenged with hPIV-1 154 days later. Interestingly, the antibody induced by hPIV-1 was cross-reactive between hPIV-1 and Sendai virus, but the generation of PIV-specific antibody was inferior to that induced by Sendai virus. Antibody rose and fell after the first inoculation with hPIV-1 and no PIV-specific IgA isotype could be identified immediately prior to the second hPIV-1 exposure. The animal with the lowest overall titer was infected after the second inoculation. This result is reminiscent of that seen in the human population, in that individuals once infected with hPIV-1, remain at risk for future hPIV-1 infections (Chanock, R. M. and McIntosh, K. “Parainfluenza viruses” in Virology, Edited by Fields, B. N. et al., Raven Press, New York, p. 963 (1990); Smith, C. B. et al., “Protective effect of antibody to parainfluenza type 1 virus”, N Engl J Med 275:1145-1152 (1966); Kingsbury, D. W. “Paramyxoviridae and their replication”, In: Virology (Eds Fields B N, Knipe D M, Chanock R M). Raven Press, New York, 1990 p. 945; Welliver, R., et al., “Natural history of parainfluenza virus infection in childhood”, J Pediatr 101:180-187 (1982)).

One explanation for the phenomenon of repeat infections with hPIV-1 in the human population is that the hPIV-1 (C35 strain) has been shown to be heat sensitive (HA degrades at 37 degrees C. (Gorman W. L., et al., “Glycosylation of the hemagglutinin-neuramimidase glycoprotein of human parainfluenza virus type 1 affects its function but not its antigenic properties”, Virology 183:83-90, 1991). Possibly, heat sensitivity thwarts the immunogenicity of hPIV-1, highlighting Sendai virus as the better vaccine.

The data described herein, in conjunction with previous work, strongly support the use of Sendai virus as a human vaccine for the following reasons:

1) Virus-specific responses generated in humans, mice and African green monkeys demonstrate strong cross-reactivity between hPIV-1 and Sendai virus. See this Example 1 and the following references: Smith, F. S. et al., “Age-related development of human memory T-helper and B-cell responses towards parainfluenza virus-type 1”, Virology 205: 453-461 (1994); Dave, V. P. et al., “Viral cross-reactivity and antigenic determinants recognized by human parainfluenza-1-specific cytotoxic T-cells”, Virology 199: 376-383 (1994)), reflecting the strong similarity between the viral proteins of the related pathogens (Lyn, D. et al., “The nucleoproteins of human parainfluenza virus type 1 and Sendai virus share amino acid sequences and antigenic and structural determinants”, J Gen Virol. 72: 983-987 (1991); Gorman, W. L. et al., “The hemagglutinin-neuramimidase glycoproteins of human parainfluenza virus type 1 and Sendai virus have high structure-function similarity with limited antigenic cross-reactivity”, Virology 175:211-223 (1990)).

2) The 461E isolate of Sendai virus has been well characterized and has been shown to be heat-stable, unlike a C35 hPIV-1 isolate, which is sensitive to degradation at 37° C. See van Wyke Coelingh, K. L., et al., “Antibody responses of humans and nonhuman primates to individual antigenic sites of the henagglutinin-neuramimidase and fusion glycoproteins after primary infection or reinfection with parainfluenza type 3 virus”, J. Virol. 64:3833-3843 (1990); Hyland, L., et al., “Respiratory virus infection of mice provokes a permanent humoral immune response”, J Virol 68:6083-6086 (1994).

3) Live virus immunizations are superior to those with inactivated virus, in that a long-lasting reservoir of memory B-cells may be elicited and maintained in the bone marrow. See Sangster, M., et al., “Distinctive kinetics of the antibody-forming cell response to Sendai virus infection of mice in different anatomical compartments”, Virology 207:287-291 (1995); Hou, S., et al., “Virus-specific CD8+ T-cell memory determined by clonal burst size”, Nature 369:652-654 (1994)). CTL responses are also long-lived (Belshe, R. B., et al., “Evaluation of a live attenuated, cold-adapted parainfluenza virus type 3 vaccine in children”, J Clin Microbiol 30:2064-2070 (1992).

For decades, multiple protocols have been tested to create attenuated forms of PIV or recombinant vectors for the purpose of vaccination. See van Wyke Coelingh, K. L., et al., “Expression of biologically active and antigenically authentic parainfluenza type 3 virus hemagglutinin-neuramimidase glycoprotein by a recombinant baculovirus”, Virology 465-472 (1987); Spriggs, M. K., “Immunization with vaccinia virus recombinants that express the surface glycoproteins of human parainfluenza virus type 3 (PIV3) protects patas monkeys against PIV3 infection”, J Virol 62:1293-1296 (1988); Spriggs, M. K, et al., “Expression of the F and HN glycoproteins of human parainfluenza virus type 3 by recombinant vaccinia viruses: contributions of the individual proteins to host immunity”, J Virol 61:3416-3423 (1987); Ryan, K. W., et al., “Separate domains of Sendai virus P protein are required for binding to viral nucleocapsids”, Virology 174:515-521 (1990)). The mouse Sendai virus represents a natural vaccine, immediately available for use without further manipulation.

### Example 2

### Safety and Immunogenicity of Intranasal Murine Parainfluenza Virus Type 1 (Sendai Virus) in Healthy Adults

Summary

Human parainfluenza virus-type 1 (PIV-1) is the most common cause of pediatric laryngotracheobronchitis (croup) and results in close to 30,000 US hospitalizations each year. Counihan, M. E. et al., “Human parainfluenza virus-associated hospitalizations among children less than five years of age in the United States”, Ped Inf Dis J 20:646-653 (2001). No effective vaccine is available. We examined murine PIV-1 (Sendai virus) as a live, xenotropic vaccine for the closely related human PIV-1 in a Phase I, dose escalation study in healthy adults. Intranasal Sendai virus was uniformly well-tolerated and showed evidence of immunogenicity in 3 of 9 vaccinees despite pre-existing, cross-reactive immunity presumably induced by human PIV-1 exposure. Results support further trials to evaluate the efficacy of Sendai virus in preventing human PIV-1 infection in infants and children.

Introduction

Human PIV-1 (hPIV-1) is a member of the paramyxoviridae family and a cause of pediatric bronchiolitis, pneumonia, and particularly of laryngotracheobronchitis, or croup. Counihan, M. E. et al., infra. (2001). In the 1960s, an inactivated, intramuscular trivalent vaccine targeting hPIV-1, -2 and -3 was prepared and tested in a pediatric population. Fulginiti, V. A. et al., “A field trial of two inactivated respiratory virus vaccines; an aqueous trivalent parainfluenza virus vaccine and an alum-precipitated respiratory syncytial virus vaccine”, Am J Epidemiol 89:435-448 (1969). Although the vaccine appeared safe, no evidence of protection was observed.

The safety of formalin-inactivated (FI) hPIV vaccine contrasted with that of both the FI-respiratory syncytial virus (RSV) and FI-measles vaccines. Recipients of the FI-RSV or FI-measles vaccine had exacerbated disease upon natural infection. Fulginiti, V. A. et al., “A field trial of two inactivated respiratory virus vaccines; an aqueous trivalent parainfluenza virus vaccine and an alum-precipitated respiratory syncytial virus vaccine”, Am J Epidemiol 89:435-448 (1969); Fulginiti, V. A. et al., “Altered reactivity to measles virus: Atypical measles in children previously immunized with inactivated measles virus vaccines”, J Am. Med. Assoc. 202: 1075-1080 (1967).

The lack of protection with FI-paramyxovirus vaccines contrasted with the results of live attenuated paramyxovirus vaccine trials. The live attenuated measles virus vaccine, for example, elicited responses that were both effective and durable, in contrast to those elicited by the inactivated vaccine.

To design a live attenuated paramyxovirus vaccine for human PIV-1, we have pursued a xenotropic or Jennerian vaccine approach. We considered murine parainfluenza virus (Sendai virus, SeV) as a good candidate for xenotropic vaccination against hPIV-1, based on findings of shared sequence homology and antigenic cross-reactivity. Lyn, D. et al., “The nucleoproteins of human parainfluenza virus type 1 and Sendai virus share amino acid sequences and antigenic and structural determinants”, J Gen Virol 72:983-987 (1991); Gorman, W. L. et al., “The hemagglutinin-neuramimidase glycoproteins of human parainfluenza virus type 1 and Sendai virus have high structure-function similarity with limited antigenic cross-reactivity” Virology 175: 211-223 (1990); Smith, F. S. et al., “Age-related development of human memory T-helper and B-cell responses towards parainfluenza virus-type 1”, Virology 205: 453-61 (1994).

We previously evaluated the capacity of SeV to protect non-human primates from hPIV-1 challenge and showed that following self-limited growth in the nasopharynx of African green monkeys, SeV elicited durable virus-specific antibody responses and uniform protection against hPIV-1 challenge. Hurwitz, J. L. et al., “Intranasal Sendai virus vaccine protects African green monkeys from infection with human parainfluenza virus-type one”, Vaccine 15: 533-40 (1997). SeV did not cause respiratory symptoms in these primates. Additionally, SeV has never been demonstrated to cause disease in humans. Our immunologic and challenge studies suggested the promise of SeV as a safe live virus vaccine for hPIV-1 in humans. As a prelude to evaluating SeV vaccine in the target population of seronegative children, we examined the safety and immunogenicity of intranasal SeV in adult volunteers.

Subjects, Materials and Methods

Nine healthy adult volunteers (2 males, 7 females; average age 28.6 years) were enrolled in a Phase I dose escalation, safety study of intranasal SeV vaccine. The protocol was reviewed by the US Food and Drug Administration (FDA) and approved by the St. Jude Children's Research Hospital and University of Tennessee Institutional Review Boards. Written informed consent was obtained from each study participant.

The vaccine consisted of unmodified live SeV (Enders strain) propagated in chick egg (Spafas Inc., Preston, Conn.) allantoic fluid. This vaccine study evaluated three doses of intranasal SeV (5×10⁵ egg infectious doses₅₀ (EID₅₀), 5×10⁶ EID₅₀ and 5×10⁷ EID₅₀) administered as a single dose to 3 cohorts consisting of 3 subjects each. The vaccine was stored at −70° C., and was thawed and diluted in sterile saline immediately prior to administration. Each dose was delivered as 0.25 ml by dropper in each nostril (total 0.5 ml) of the supine volunteer. For 28 days after vaccination, subjects were evaluated for the development of respiratory symptoms (by evaluation or questionnaire) and were requested to complete a daily diary card to record any signs or symptoms. Subjects returned to clinic for examination on days 2, 4, 7, 10, 14, 28, 182 and 365. Blood was obtained on days 7, 14, 28, 182 and 365 for safety studies and for analysis of antibodies (by ELISA) to SeV and to hPIV-1. Nasal swabs obtained in the first month (days 0, 2, 4, 7, 10, 14 and 28) following vaccination were tested for the presence of vaccine virus and for vaccine elicitation of specific mucosal antibody.

To detect virus-specific antibody binding responses, ELISAs were performed by coating 96-well plates with purified, disrupted SeV or hPIV-1 (0.5 μg) as a source of antigen. After non-adsorbed virus was removed, well surfaces were blocked (1.0% BSA in PBS) and washed with PBS containing 0.05% Tween 20 (PBST). Plasma was diluted 1:1000 and applied to wells (50 ul/well) for overnight incubation (24° C.). Wells were washed in PBST, and developed with alkaline phosphatase-conjugated goat anti-human immunoglobulin and p-nitrophenyl phosphate. Nasal swab samples were diluted 1:5 in PBS and tested for the presence of specific antibody by ELISA as above, but with isotype-specific (IgG or IgA) secondary goat anti-human antibodies (Southern Biotechnology Associates, Birmingham, Ala.).

To detect hPIV-1 neutralizing activity, virus (100 pfu hPIV-1) was incubated with serum for 1 hr and the mixture was then inoculated in duplicate onto 6-well plates with confluent monolayers of LLC-MK2 cells for 1 hr. Wells were washed and cells were then grown in DMEM with 10% FCS (24 hrs, 34° C.). Cells were methanol-fixed and stained with a cocktail of hPIV-1-specific mouse monoclonal antibodies. Secondary staining was performed with horseradish peroxidase-conjugated anti-mouse IgG (Bio-Rad, Hercules, Calif.) and developed with DAB (3,3′-diaminobenzidine; Sigma-Aldrich, St. Louis, Mo.). Infected cells were visualized as dark brown or purple cells.

Results

SeV vaccine was uniformly well-tolerated without any reactions. None of the subjects developed any respiratory symptoms or laboratory abnormalities (Table 2 below). SeV was not detected from nasal cavity samples by virus culture or by egg inoculation.

TABLE 2
Laboratory parameters of vaccinees following SeV vaccination.

| | | 7 d Post- | 14 d Post- | 28 d Post- |
|---|---|---|---|---|
| Laboratory Test | Pre-vaccine | vaccine | vaccine | vaccine |
| Hgb, gm/dL | 13.3 | 13.4 | 13.7 | 13.6 |
| Platelets(cells × 10³/ | 259 | 232 | 230 | 240 |
| mm³) | | | | |
| WBC, cells/mm³ | 5400 | 5600 | 5400 | 5200 |
| % Neutrophils | 61 | 60 | 62 | 60 |
| % Monocytes | 8 | 8 | 9 | 8 |
| % Lymphocytes | 28 | 26 | 27 | 26 |
| % Eosinophils | 1 | 2 | 2 | 2 |
| Total bilirubin, mg/dL | 0.4 | 0.5 | 0.3 | 0.4 |
| ALT, units/L | 20 | 19 | 19 | 26 |
| AST, units/L | 16 | 16 | 18 | 18 |
| Creatinine, mg/dL | 0.7 | 0.8 | 0.8 | 0.8 |
| Amylase, units/L | 51 | 51 | 59 | 54 |

Median laboratory parameters for each time point (n = 9).

All study subjects demonstrated pre-existing antibody directed against SeV. This antibody is presumably the result of previous hPIV-1 infection—the presence of shared antigenic epitopes between murine SeV and human PIV-1 causes hPIV-1-induced antibody to score positive in SeV-based ELISAs. Despite this pre-existing antibody, intranasal SeV inoculation induced a significant (≧4-fold, range 4.5-24 fold) increase in serum antibody titers measured against SeV, starting approximately 2 weeks post vaccination in 3 of the 9 vaccinated subjects. The 3 subjects with significant increase in specific antibody titers received different vaccine doses (5×10⁵ EID₅₀ [n=1] and 5×10⁷ EID₅₀ ([n=2]), and did not differ from other vaccinees in their baseline serum titers (all subjects were positive at 1:1000 serum dilution at baseline). In all cases, antibody titers to SeV correlated with titers to hPIV-1. Elevated titers observed among the subset of responders returned to pre-existing baseline levels by 6-12 months following vaccination.

Nasal swabs from vaccinees with positive serum responses were also tested and pre-existing responses were again detected from these subjects (at 1:5 dilution nasal swab sample). Intranasal SeV elicited a boost in IgG and IgA virus-specific antibodies in nasal swabs post-vaccination among these individuals.

As a final assessment of the quality of the immune response elicited by intranasal SeV, serum from one of the 3 responding volunteers (Vac 012) was tested for the capacity to neutralize hPIV-1 in vitro. Paralleling the uniform presence of hPIV-1 specific binding antibody among adults, hPIV-1 neutralizing antibody was also evident in baseline serum (detectable from Vac 012 at 1:64 serum dilution). Comparison of serum obtained pre- and one month post-vaccination demonstrated a marked increase in serum neutralizing capacity (reduction in the number of infected cells in vitro) following vaccination (serum dilution 1:512).

Discussion

The present report demonstrates the potential for SeV to serve as a naturally attenuated live virus vaccine for hPIV-1. Intranasal SeV was well-tolerated, caused no respiratory or allergic symptoms and was not recovered from the nasal passages of seropositive adults. Each of the three escalating doses was equally well tolerated. The observation that intranasal SeV can boost serum and mucosal antibody responses among immunologically experienced adults serves simply as a proof of principle that intranasal SeV can be immunogenic in humans. The ultimate target population for a SeV vaccine will be immunologically naïve children. Results from the current study reinforce the shared antigenicity of murine and human PIV-1: human responses elicited by intranasal SeV can bind and neutralize hPIV-1 in vitro. Such in vitro responses correlated with protection from hPIV-1 challenge in African green monkeys. Hurwitz, J. L. et al., “Intranasal Sendai virus vaccine protects African green monkeys from infection with human parainfluenza virus-type one”, Vaccine 15: 533-40 (1997).

It is likely that SeV will also prove safe in seronegative humans due to host-range restriction resulting from evolution in mouse versus man. SeV was originally identified in 1952 from mice naturally infected with SeV, who were then inoculated with human samples. Ishida, N. and Homma, M., “Sendai virus”, Adv Virus Res 23: 349-383 (1978). Despite abundant contact between mice and children, there has been no confirmed case of SeV human disease since this original discovery of the virus. Our analyses of nasal tissues by swab samplings showed that SeV can replicate in the upper respiratory tract (URT) of seronegative African green monkeys for approximately 4 days. Hurwitz. J. L., infra. (1997). This was confirmed by another group using bronchoalveolar lavage (see Skiadopolous, M. H. et al., “Sendai virus, a murine parinfluenza virus type 1, replicates to a level similar to human PIV1 in the upper and lower respiratory tract of African green monkeys and chimpanzees”, Virology 297:153-160 (2002)) This bronchoalveolar lavage procedure may not discriminate between URT and lower respiratory tract samplings. Bartlett, J. G. et al., “Should fiberoptic bronchoscopy aspirates be cultured?”, Am Rev Respir Dis 114: 73-78 (1976).

Neither we nor others identified symptoms among inoculated animals. Hurwitz. J. L., infra. (1997); Skiadopolous, M. H. et al (2002). We note that most clinically effective viral vaccines exhibit infection without disease, a characteristic that we expect to confirm for SeV with our continued clinical studies.

A safe intranasal paramyxovirus vaccine holds great appeal: oral or nasal vaccines obviate requirement for sterile needles and syringes, and nasal vaccine administration is particularly effective at eliciting local IgA responses. Importantly, the latter has been correlated with beneficial, non-inflammatory responses toward common respiratory viruses. Russell. M. W. et al., “Strategies of immunization against mucosal infections”, Vaccine 19: S122-S127 (2000).

Our finding that intranasal SeV boosts mucosal IgG and IgA responses in this study thus reinforces the potential utility of this vaccine. Additionally, as a live virus vaccine, intranasal SeV is expected to elicit cellular immune responses, which are likely to prove important in durable protection from hPIV-1 challenge. Lyn, D. et al. infra. (1991); Slobod, K. S. and Allan, J. E., “Parainfluenza type 1 virus-infected cells are killed by both CD8+ and CD4+ cytotoxic T cell precursors” Clin Exp Immunol 93: 363-369 (1993).

An effective paramyxovirus vaccine should also elicit neutralizing antibody. The failure of FI RSV vaccine to induce neutralizing antibody responses (while inducing CD4+ T cell responses) may have been critical to the pathogenesis of exacerbated pulmonary disease following natural RSV infection in young vaccine recipients. Fulginiti, V. A. et al, infra (1969). Accordingly, SeV-activation of hPIV-1-specific neutralizing responses in this study constitutes an important vaccine milestone. Live attenuated paramyxovirus vaccines, which can induce effective and long-lasting B, CD4+ and CD8+ T cell responses, circumvent many concerns regarding FI viral vaccines.

In conclusion, SeV has proven safe in a limited adult trial, designed as a forerunner to forthcoming studies in children. Success in future clinical studies is expected to prove SeV an effective vaccine in the prevention of hPIV-1 mediated croup in infants and children.

Various publications, patent applications and patents have been cited herein, the disclosures of which are incorporated by reference in their entireties.

1-5. (canceled)

6. A method for protecting a human subject against systemic human parainfluenza virus (hPIV) infection comprising administering to the subject an effective immunizing amount of a composition comprising a Sendai virus and a pharmaceutically acceptable carrier.

7. The method of claim 6 wherein said composition is administered to the upper respiratory tract.

8. The method of claim 6 wherein said composition is administered in a form selected from the group consisting of a spray, one or more droplets and an aerosol.

9. The method of claim 6 wherein said composition is administered to said subject by a means selected from the group consisting of intranasal administration, intravenous administration, intramuscular administration, subcutaneous administration, intradermal administration, and administration to mucous membranes.

10. The method of claim 6 wherein said composition elicits an hPIV-1 specific immune response in the group of cells consisting of B-cells or T-cells (CD4+ and/or CD8+ T-cells), or a combination thereof.

11. The method of claim 6 wherein said subject is a human.

12. The method of claim 11 wherein said human is less than 10 years old.

13. The method of claim 12 wherein said human is less than 5 years old.

14. The method of claim 13 wherein said human is less than 1 year old.

15. The method of claim 6 herein said effective immunizing amount is between 1×10⁵-1×10⁸ plaque forming units (pfu).

16. A method for enhancing the immune response of a subject previously infected with hPIV to a subsequent hPIV infection comprising administering to the subject an immunizing amount of a composition comprising a Sendai virus and a pharmaceutically acceptable carrier.
